(19) United States
(12) Patent Application Publication
(10) Pub. No.: US 2026/0061196 A1
(43) Pub. Date: Mar. 5, 2026

(54) TECHNIQUES FOR A WEARABLE ELECTRONIC STIMULATING DEVICE

(71) Applicant: Relief Technologies AS, Oslo (NO)

(72) Inventors: Fredrik F. Hassel, Oslo (NO); Henrik Børsting Jacobsen, Oslo (NO)

(21) Appl. No.: 19/319,605

(22) Filed: Sep. 4, 2025

Related U.S. Application Data
(60) Provisional application No. 63/737,492, filed on Dec. 20, 2024.

(30) Foreign Application Priority Data
Sep. 4, 2024 (NO) 20240902

(51) Int. Cl. A61N 1/36; A61M 21/00; A61M 21/02; G16H 20/70; G16H 40/67

(52) U.S. Cl. CPC A61N 1/36021; A61M 21/02; A61N 1/36031; G16H 20/70; G16H 40/67; A61M 2021/005; A61M 2021/0088; A61M 2205/3553

(57) ABSTRACT

The disclosure relates to a method of operating a wearable electronic stimulation device. The method can include providing and arranging the wearable electronic vagus nerve stimulating (VNS) device and applying vagus nerve stimulation using the VNS device. The method can also include monitoring and recording a user's physical symptoms and providing to the user behavior instructions. The method can also include combining the vagus nerve stimulation with the monitored and recorded physical symptoms, and/or the behavior instructions to generate a personalized treatment plan for managing the user's chronic pain.

### CROSS-REFERENCE TO RELATED APPLICATION

This application claims the priority benefit of Norwegian patent application No. 20240902 filed Sep. 4, 2024, and U.S. Patent Application No. 63/737,492 filed Dec. 20, 2024, which are incorporated by reference herein in their entirety.

### TECHNICAL FIELD

The present disclosure relates to a wearable electronic stimulating device, a method for operating a wearable electronic stimulating device and a system for operating a wearable electronic stimulating device.

### BACKGROUND ART

Chronic muscle pain is a major societal problem with no effective treatment, affecting approximately 30% of the adult population globally. Nearly 80% receive no pain relief from any available treatments including medications. It is estimated that 47.5% of all long-term sick leave is caused by chronic pain.

Chronic muscle pain conditions are prevalent and often difficult to manage effectively with conventional therapies. The lack of effective treatments has severe consequences and, as such, new options for pain relief are highly sought after and commercially viable.

Problems with the solutions of the prior art are that only 2 out of 10 get any relief from pain medications, and the rest only get negative side-effects, surgery does not work, and physical therapy and psychology only helps a few, with physical and cognitive change being slow, challenging and expensive. Patients do not want to take drugs because of side-effects and addiction.

In the search for lasting pain relief, stimulating the vagus nerve (Vagus Nerve Stimulation-VNS) has been seen as a viable option. The vagus nerve (the 10th cranial nerve) innervates multiple internal organs and integrates sensory, motor, and autonomic information by four vagal nuclei. It can alleviate pain through many pathways but the most promising is through altering descending modulation of pain in the nucleus tractus solaritii which is enervated by vagal efferents.

One main obstacle causing VNS to not be widely implemented is that surgical implementation comes with high costs and high risks to patients' health. However, non-invasive VNS delivery systems do not require surgery and these systems have improved the safety and tolerability of VNS, making it more accessible and feasible. One such non-invasive system, Transcutaneous Vagus Nerve Stimulation (t-VNS), uses the auricular branch of the vagus nerve, which extends to the outer ear. This means that the nerve branch can be stimulated through the skin (transcutaneous) with electrical impulses. Intensity, pulse duration and frequency of the t-VNS stimulation are targeted to induce signaling from the A-fibers of this auricular branch. Auricular nerve fibers project to brain structures, which are involved in the descending inhibitory modulation of pain.

However, current t-VNS stimulators have a low hit ratio with limited efficiency and usability. There are no t-VNS stimulators in the marketplace that provide a sufficiently efficient product for pain relief. Some inadequate products are offered at a high price range.

Non-invasive treatment methods for chronic pain using an integrative approach has been proposed, comprising one or more of:
interoceptive exposure (somatic tracking and provocative testing) to expose for painful movement in a safe way through visualization as well as monitor and evaluate physical symptoms and pain triggers:
   pain-relieving hypnosis to alleviate pain perception and improve relaxation; and
   meditation practices including Mindfulness-Based Stress Reduction (MBSR), pranayama breathing, and non-directive meditation to promote relaxation and enhance treatment efficacy.

Chronic pain conditions are prevalent and often difficult to manage effectively with conventional therapies. Current treatments may include physical therapy, medication, cognitive-behavioral therapy, or invasive procedures, which may bring limited relief or unwanted side effects.

Problems include that prior art methods and devices lack adaptability, flexibility and efficiency, and are cumbersome to use/ill fitted and expensive.

There is thus a need for improved device, method and system for relieving pain sensation.

### SUMMARY

It is an object of the present disclosure to mitigate, alleviate or eliminate one or more of the above-identified deficiencies and disadvantages in the prior art and solve at least the above-mentioned problem. A novel device is presented for transcutaneous auricular vagus nerve stimulation (taVNS), to modulate nerve activity and reduce pain.

According to an aspect of the present invention there is provided a wearable electronic stimulation device for transcutaneous auricular vagus nerve stimulation, taVNS, comprising: a distal/first portion comprising one or more electrode modules arranged on an outer surface of the first portion, the first portion configured to be positioned at least partially within the cymba concha of a user's ear when the device is worn in the user's ear, and each electrode module having an outer curved surface configured such that the one or more electrode modules contact the skin of the cymba concha when the device is worn in the user's ear; and a proximal/second portion coupled to the first portion, the second portion comprising or being remotely connected to one or more of: a control module for controlling the one or more electrode modules; and a power source for powering the control module and the one or more electrode module.

In some embodiments, the first portion has a resilient C-shape and/or a cylindrical tapering towards a distal end configured to fit inside the cymba concha of an ear when the wearable electronic stimulation device is in use, for providing contact towards the inside curve of the antihelix and the cymba concha of the ear.

In some embodiments, the cylindrical tapering of the first portion comprises an external curvature intended for maximizing the contact with the cymba concha of a user's ear.

In some embodiments, the second portion is coupled to the first portion by a resilient member.

In some embodiments, the second portion is configured to be positioned at least partially within the cavum concha of a user's ear when the device is worn in the user's ear.

In some embodiments, the resilient member is configured to bias the first portion and the second portion to positions in which the electrode modules are pressed against the skin of the cymba concha when the device is worn in the user's ear.

In some embodiments, the resilient member is deformable to allow the device to be mounted in a user's ear, with the first portion at least partially positioned within the cymba concha and the second portion at least partially positioned within the cavum concha.

In some embodiments, the second portion comprises one or more additional electrode modules arranged on an outer surface of the second portion and having an outer curved surface configured such that the one or more additional electrode modules contact the skin of the cavum concha when the device is worn in the user's ear, the one or more additional electrode modules connected to the control module for control by the control module and connected to the power source for being powered by the power source.

According to another aspect of the present invention there is provided a wearable electronic stimulating device for transcutaneous auricular vagus nerve stimulation, taVNS, comprising: a distal portion comprising one or more distal electrode modules, a proximal portion comprising or being remotely connected to one or more of: a control module and a power source for powering the control module and the distal electrode modules, the distal portion having a cylindrical tapering towards the distal end resilient C-shape configured to fit inside the cymba concha of an ear when the wearable electronic stimulating device is in use, for providing contact towards the inside curve of the antihelix and the cymba concha of the ear, and each distal electrode module having an outer curved surface adapted to, and arranged in, the outside surface of the distal portion. The configuration of the distal electrode modules is such that they are positioned for being brought in contact with the cymba concha of an ear when the wearable electronic stimulating device is in use.

The resilient C-shaped portion may be deformable, thereby enabling adaptation of its shape to various anatomical shapes of a user's ear.

An advantage of the device according to the above aspects and the rest of the present disclosure is that it provides optimal contact between the electrode modules and the cymba concha of an ear of a user when in use. Further, the device adapts effectively to individual anatomical variations of the ear when the device is worn.

A further advantage of the device according to the present disclosure is that the device may be self-sustained in that it may comprise its own power and controller, and hence does not involve handling multiple entities, external cabling and additional separate components. The power source may be rechargeable, and the device may comprise a communication module for wireless communication with external devices such as a smartphone.

According to some embodiments the proximal portion of the wearable electronic stimulating device comprises one or more proximal electrode modules, the proximal electrode module being controlled by the control module, and the one or more proximal electrode modules having an outer curved surface adapted to, and arranged in, the outside surface of the proximal portion at a position for being brought in contact with the cavum concha of an ear when the wearable electronic stimulating device is in use.

The proximal electrode modules may thus be shaped to optimize/maximize the contact area with the cavum concha.

According to some embodiments the cylindrical tapering of the distal portion comprises an external curvature intended for maximizing the contact with the cymba concha of a user's ear. Enhanced contact along the cymba concha improves the gripping force and retention of the wearable device within the ear, maintaining stable positioning during user movement and activities, and ensures consistent contact for the distal electrode modules.

According to some embodiments any of the electrode modules is electrically paired with another of the electrode modules, and the control module is configured to output a controlled voltage and/or current signal across the paired electrode modules. The strength and pattern of the signal may also be outputted according to a predefined scheme.

Such pairing configurations allow greater flexibility in adjusting signal patterns, directionality, and electrode positioning. By electrically pairing multiple electrode modules, it becomes possible to triangulate the electrical stimulation signals within specific regions of the ear, optimizing the precision of stimulation and enhancing user experience and therapeutic outcomes. This triangulation capability enables targeted signal delivery, more precise modulation of nerve pathways, and reduced unintended stimulation of surrounding tissue, thereby improving comfort and effectiveness. The amplitude, frequency, and pattern of these signals may be executed according to predetermined protocols designed for specific therapeutic applications or dynamically adjusted in response to user feedback or physiological measurements.

According to some embodiments the control module is configured to control the pairing configuration of the electrode modules, for example dynamically.

According to some embodiments, the control module may be designed to deliver predetermined currents to the electrode modules following specific patterns and polarities for predefined pairing schemes, thereby enabling dynamic in situ adjustments as well as execution of predefined stimulation protocols.

Thus, enabling both in situ dynamic changing and predefined configuration of pairing configuration and signal patterns to be executed.

According to some embodiments the wearable device comprises: a heart rate sensor.

This facilitates dynamic customization of electrode pairing, signal patterns, and/or signal intensities based on real-time user physiological feedback.

According to some embodiments the heart rate sensor is a Photoplethysmography, PPG, sensor, and the sensor is arranged behind a sensor window arranged in the proximal portion of the wearable electronic stimulating device.

According to some embodiments one or more of the electrode modules outer surface the wearable comprises an array of multiple spikes, or multiple microstructures that may form spikes.

The surface of the skin is composed of a hard layer of non-conducting dead cells, the epidermal stratum corneum. The spikes can penetrate through the epidermal stratum corneum of the ear to provide a better transmission of electrical pulses between the electrode modules and the target area for stimulation. According to some embodiments the spikes have a height h between about 0.1 and about 0.9 mm, and more advantageous between about 0.2 and about 0.6 mm, and most advantageous between about 0.3 and about 0.5 mm.

The form of the spikes is advantageously solving the trade-off of being comfortable enough for the user to use the wearable electronic stimulating device, and for the signals to be properly transferred to the ear.

According to some embodiments the spikes are arranged with at least about 0.2 mm distance between spikes, or more advantageous with at least about 0.4 mm distance between spikes, or most advantageous more than about 0.6 mm distance between spikes.

According to additional embodiments, each outer curved surface comprising an array of spikes comprises a convex circular-arc profile. In some embodiments, the outer curved surface defines a circular-arc profile having a chord width W and an arc height H. The width W may be selected to correspond to the cavum concha width, and the height H may be selected to conform to the cavum wall.

In some examples, the width W may be in the range of about 14 mm to about 21 mm, preferably about 16 mm to about 19 mm. In some examples, the height H may be in the range of about 2 mm to about 3 mm, preferably about 2.2 mm to about 2.8 mm. A width-to-height ratio W:H may be in the range of about 5:1 to about 10:1, preferably about 6:1 to about 9:1.

In some embodiments, the circular-arc profile has a radius of curvature R given by the relationship R=W²/8H+H/2. The radius of curvature R may be in the range of about 10 mm to about 25 mm, preferably about 14 mm to about 20 mm.

The spikes in the array may have a substantially constant height, so that the tips of the spikes also follow the convex arc profile of the outer curved surface.

This low arc profile gives the electrode module surface a distinctive cross-sectional profile which maximizes effective contact surface area while minimizing penetration depth, thereby enhancing electrical stimulation without causing discomfort or pain. The gentle curvature and rounded apex ensure shallow, controlled skin penetration specifically targeting the epidermal stratum corneum within the cymba concha, closely matching its anatomical contours. The optimized arc shape provides precise control over stimulation currents, enabling targeted therapeutic effects with enhanced precision. Furthermore, the increased friction provided by the unique surface area of the low arc shape improves device stability and retention within the ear during movement, substantially reducing the risk of displacement. The absence of sharp edges minimizes potential micro-injury, irritation, or abrasion to the user's skin, significantly enhancing user comfort and safety. Additionally, this spike array geometry streamlines manufacturing processes, allowing higher precision and consistency across batches. The larger contact surface of the arc shape reduces impedance at the electrode-skin interface, thereby improving electrical efficiency, prolonging battery life, and optimizing device performance.

According to some embodiments the spikes has a conical tapering towards the top, and the cone top of the spikes are configured to be able to penetrate the epidermal stratum corneum or the outer protective layer of the skin of a user's ear.

According to some embodiments the number of spikes in any of the distal electrodes are more than 20, or more advantageous more than 30, or most advantageous more than 40.

According to some embodiments the number of spikes in any of the proximal electrodes are more than 40, or more advantageous more than 60, or most advantageous more than 80.

According to some embodiments the wearable comprises one or more sensors, being any of: temperature sensor, magnetic and/or electric field sensor, humidity sensor, pressure sensor, accelerometer and gyroscope sensor, electrodermal activity and heart rate sensor.

According to some embodiments the control module is further configured to use input from the sensors to set the output voltage level over the electrode module pairs.

According to some embodiments the distal portion is a replaceable portion the wearable comprises a distal connector device and electrical connectors for quick lock/release from the proximal portion, wherein the proximal portion the wearable comprises a corresponding proximal connector device and electrical connectors for cooperating with the distal connector device and electrical connectors.

The ability to be replaced ensures that the portion most likely to wear out, easily can be replaced. This is also a feature ensuring that a variation of distal portion C-shapes may be shipped, and user can choose which fits the best. It is also a way to solve variations in electrode module characteristics and positions, when distal portion C-shapes have different configurations and may be changed to achieve variations in the properties of the distal portion C-shape when in use.

According to some embodiments the electrical connectors comprise the distal and proximal connector device.

The advantage of this feature is that there are less components in play for facilitating connection and signal transfer.

According to some embodiments the connector devices comprise one or more of: a magnet, a spring loaded snaplock, a bayonet type connector, a threaded type connector, male/female alignment pin, and Registered jack, RJ.

According to some embodiments the proximal portion further comprises a speaker device arranged for outputting sound, the speaker device being electrically connected to and controlled by the control module.

This provides for using the device in combination with audio treatment. Another functionality of the speaker device is to output music and/or speech, such as use instructions, status and progress, or other.

According to some embodiments the wearable electronic stimulating device comprises a communication device for enabling communication between the electrode control module and a remote control module.

According to some embodiments the electrodes modules and the distal and proximal portion provides a waterproof outer surface, wherein the electronic components inside the distal and proximal portions are connected in a dry environment.

According to another aspect of the present invention there is provided a method for operating a wearable electronic stimulating device, comprising the steps: providing a wearable electronic stimulating device according to any of the above aspects and embodiments, providing a current output pattern for the control module, and arranging the wearable electronic stimulating device for outputting the voltage/current according to the provided output pattern.

According to another aspect of the present invention there is provided a system for operating a wearable electronic stimulating device comprising: one or more wearable electronic stimulating device according to any of the above aspects and embodiments, and a remote control module for controlling the one or more wearable electronic stimulating device.

Effects and features of the above aspects are to a large extent analogous to and compatible with each other. Embodiments mentioned in relation to one aspect are largely compatible with the other aspects.

It is a further object of the present disclosure to describe a novel method of treatment for pain management through the combination of pain reprocessing techniques such as interoceptive exposure (including somatic tracking and provocative testing), pain-relieving hypnosis based on advanced techniques for pain management, vagus nerve stimulation, VNS, heart rate variability, HRV, tracking, and various meditation practices, delivered through a dedicated platform such as an iOS and Android application.

Integrative approaches that marry physical, psychological, and neuromodulation techniques hold promise for more effective management of chronic pain.

According to another aspect there is provided a method for treatment of a user's chronic pain conditions and operating a wearable electronic stimulating device, comprising the steps:
providing and arranging a wearable electronic vagus nerve stimulating, VNS, device,
applying vagus nerve stimulation using the VNS device, and
   monitoring and recording the user's physical symptoms, and/or
   providing to the user behaviour instructions,
combining the vagus nerve stimulation with the monitored and recorded physical symptoms, and/or the behaviour instructions, for generating a personalized treatment plan for managing the user's chronic pain.

According to some embodiments, according to wherein the VNS is a transcutaneous auricular VNS, taVNS, device according to arranged on a user's ear.

According to some embodiments, the vagus nerve stimulation is controlled by: providing a current output pattern for the control module; and arranging the wearable electronic stimulating device for outputting a voltage/current according to a provided output pattern.

According to some embodiments, the monitoring and recording physical symptoms the method comprises using interoceptive exposure where the user complete standardized movements, visualisations and actions to provoke symptoms in a controlled manner logging one or more of, but not limited to: pain intensity, frequency, and duration.

According to some embodiments, the monitoring and recording physical symptoms the method comprises monitoring a measure of heart rate variability, HRV, to track physiological responses and optimize treatment interventions, wherein the monitored data is used to tailor one or more of, but not limited to: hypnosis, meditation, and taVNS protocols to individual physiological states.

According to some embodiments, wherein the interoceptive exposure comprise somatic tracking and/or provocative testing.

According to some embodiments, the providing to the user behavior instructions comprise one or more of, but not limited to: providing pain-relieving hypnosis sessions, and integrating meditation practices to provoke symptoms in a controlled manner.

According to some embodiments, the providing pain-relieving hypnosis sessions comprise employing techniques including one or more of: guided imagery, progressive muscle relaxation, deep breathing, and cognitive restructuring to alter pain perception and/or promote relaxation.

According to some embodiments, the integrating meditation practices comprise one or more of, but not limited to: Mindfulness-Based Stress Reduction, MBSR, techniques, pranayama breathing exercises, and non-directive meditation, and personalized meditation plans: to promote and/or enhancing one or more of: managing stress, relaxation, pain relief, safety, mental clarity, enhance treatment efficacy, and a comprehensive and integrative approach to pain management.

According to some embodiments, the method comprises: providing a dedicated computer platform, and configuring the computer platform by providing one or more applications running on the computer platform for providing one or more of, but not limited to: monitoring and recording the user's physical symptoms, controlling the wearable electronic vagus nerve stimulating device, and providing to the user behavior instructions.

According to some embodiments, the computer platform comprising a mobile computer device.

According to some embodiments, the method comprises the steps:
integrating all treatment components within applications on the computer to execute one or more of, but not limited to: generating personalized treatment plans: employing algorithms to analyze interoceptive exposure data and dynamically adjusting hypnosis sessions; control the output of the deployed VNS device; and instructing on meditation practices; and
based on user-specific needs and ongoing feedback, providing one or more of, but not limited to: real-time instructions: feedback; and progress tracking to enhance treatment adherence and effectiveness.

According to some embodiments, the interoceptive exposure using one or more of, but not limited to:
standardized scales, wherein standardized scales being one of, but not limited to, the Visual Analog Scale, or the Numeric Rating Scale; and
visual analytics such as trend graphs and heat maps;
to help users and clinicians identify pain triggers and understand pain progression.

According to some embodiments, the provocative testing component of the interoceptive exposure including one or more of but not limited to: detailed instructions: video demonstrations; and prompts for real-time symptom feedback to facilitate reproducibility of pain triggers.

According to some embodiments, the pain-relieving hypnosis sessions are based on pain management techniques comprising customized session parameters, the session parameters being one or more of, but not limited to: duration: background sounds; and focus areas of the body or somatic topography, tailored to the user's specific pain conditions and preferences.

According to some embodiments, the method comprises the step:
controlling the VNS device using stimulation protocols, wherein the stimulation protocols are based on one or more of, but not limited to:
   clinical guidelines, with real-time monitoring, and
   individualized treatment needs by adjusting one or more of, but not limited to: intensity: frequency; and duration, and
controlling the VNS device comprising:
   providing a voltage/current output pattern for the control module, and
   arranging the VNS device on the user for outputting a voltage/current according to a provided output pattern.

According to some embodiments, the method comprises: enhancing user engagement and adherence by one or more of, but not limited to: push notifications: reminders: progress tracking: motivational support; and daily health tips, to enhance user engagement and adherence.

According to some embodiments, the method comprises providing secure cloud-based system comprising the computer platform, and the secure cloud-based system ensures one or more of, but not limited to: data privacy: facilitating regular updates; and allowing for remote monitoring and telehealth integration by healthcare providers.

The present disclosure will become apparent from the detailed description given below: The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### Terminology

Electrodermal activity (EDA: sometimes known as galvanic skin response, or GSR) refers to the variation of the electrical conductance of the skin in response to sweat secretion. This can be done for example with EDA sensors that measure the electrical signal recorded by electrodes applied to the skin.

### Abbreviations

VNS-Vagus nerve stimulation
aVNS-Auricular vagus nerve stimulation
t-VNS-Transcutaneous vagus nerve stimulation
taVNS-Transcutaneous auricular vagus nerve stimulation
RMSSD-root mean square of successive differences
MBSR-mindfulness-based stress reduction

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings (some of which includes contour lines for better understanding the form of the devices).

FIG. 1 illustrates various sections of a human ear.

FIG. 2 illustrates major nerve paths in an ear.

FIG. 3A illustrates a first embodiment of the device according to the present disclosure.

FIG. 3B illustrates the device from FIG. 2A arranged in a user's ear.

FIG. 3C illustrates a cross-section view of the distal portion of the C-shape.

FIG. 3D illustrates the proximal part of the device seen from the connecting surface side highlighting the arc-form of the electrode module.

FIG. 3E illustrates the distal part of the device seen from the connecting surface side.

FIG. 3F illustrates a detail of the distal portion highlighting the arc-form of the electrode modules.

FIG. 3G illustrates the connecting members of the proximal portion interacting with the distal portion.

FIG. 3H illustrates the PCB and components arrangement of the proximal portion interacting with the distal portion

FIG. 3I shows an exploded view of the components of one embodiment of the proximal portion.

FIG. 3J shows an exploded view of the components of one embodiment of the distal portion.

FIG. 3K illustrates the circular-arc profile of the electrode module.

FIGS. 4A-4C illustrates the device according to a second embodiment of present disclosure seen from front, above and below respectively.

FIG. 5 shows modules of a high-level system according to present disclosure.

FIG. 6 shows a use scenario of present disclosure.

FIG. 7 illustrates how signal pulses may be modified/defined according to present disclosure.

FIG. 8 shows a flow diagram showing the integrated services provided by embodiments of the present disclosure.

FIG. 9 shows a cloud based system according to embodiments of the present disclosure.

### DETAILED DESCRIPTION

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

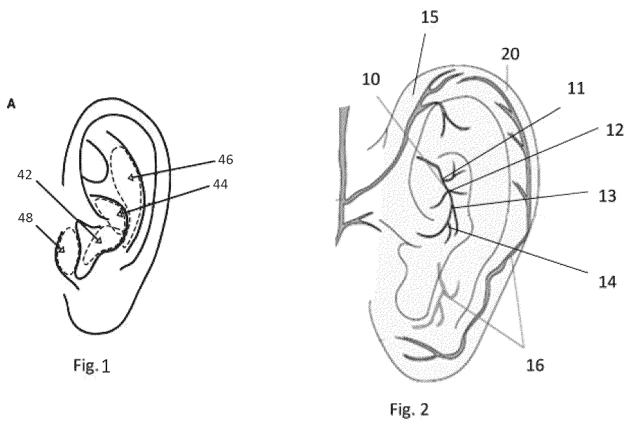

The present disclosure discloses a device, method and system for non-invasive activation of the auricular branch of the vagus nerve 10. As shown in FIG. 2, the auricular branch of the vagus nerve 10 is found in the outer ear, and as such an optimal site for stimulation where you may hit the auricular branch of the vagus nerve with precision.

FIG. 1 shows the anatomy of the human ear, showing the locations of the cavum conchae 42, cymba conchae 44, antihelix 46 and tragus 48. The novel device and method provide the possibility to optimize the hit rate of the pain relief by stimulating several areas within the cavum conchae 2 and cymba conchae 4.

A 4-year trial conducted by the inventors of present disclosure was performed using prior art methods, showing first evidence of t-VNS relieving pain in Fibromyalgia syndrome (FMS). Trial data revealed three essential limitations in the t-VNS apparatus pertaining to stimulation areas, strength and comfort, reducing its ability to alleviate pain.

The present disclosure reveals that the stimulation of multiple points of the vagus nerve in the ear will increase efficiency. This may be caused by the fact that the nature and location of where the vagus nerve is most prominent differs from one to another individual.

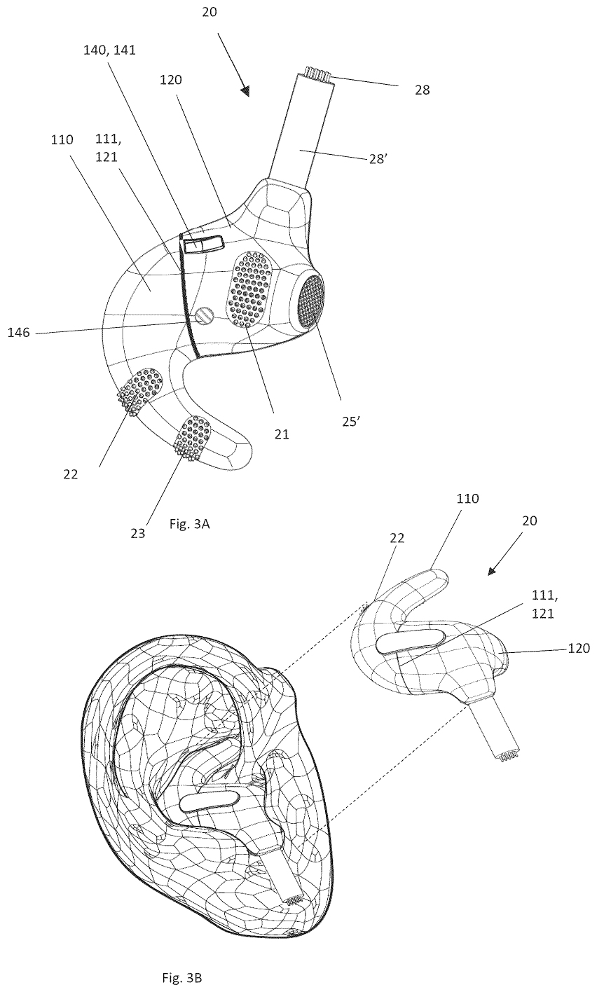

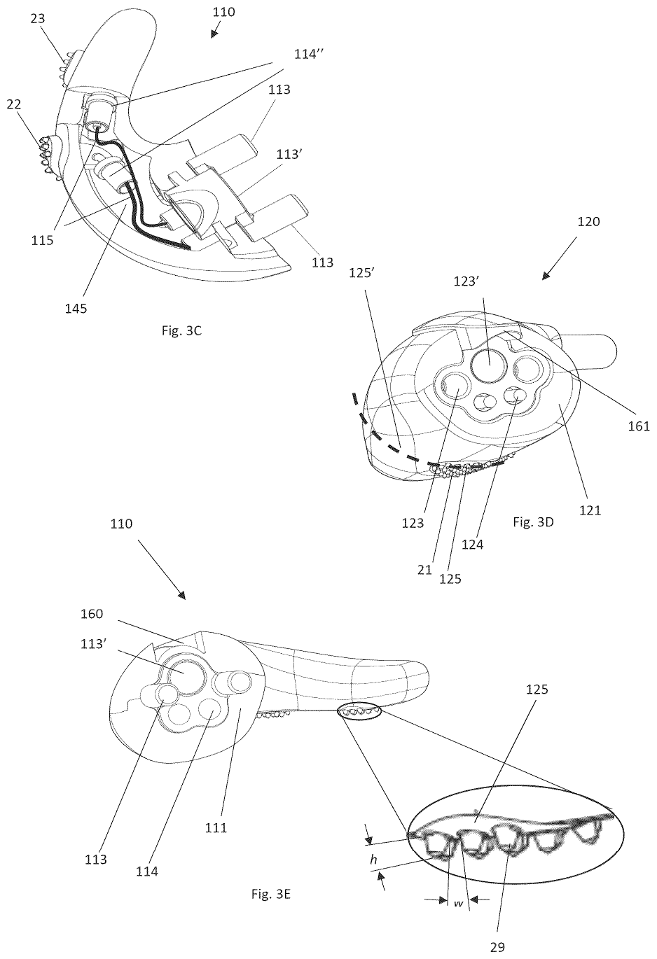

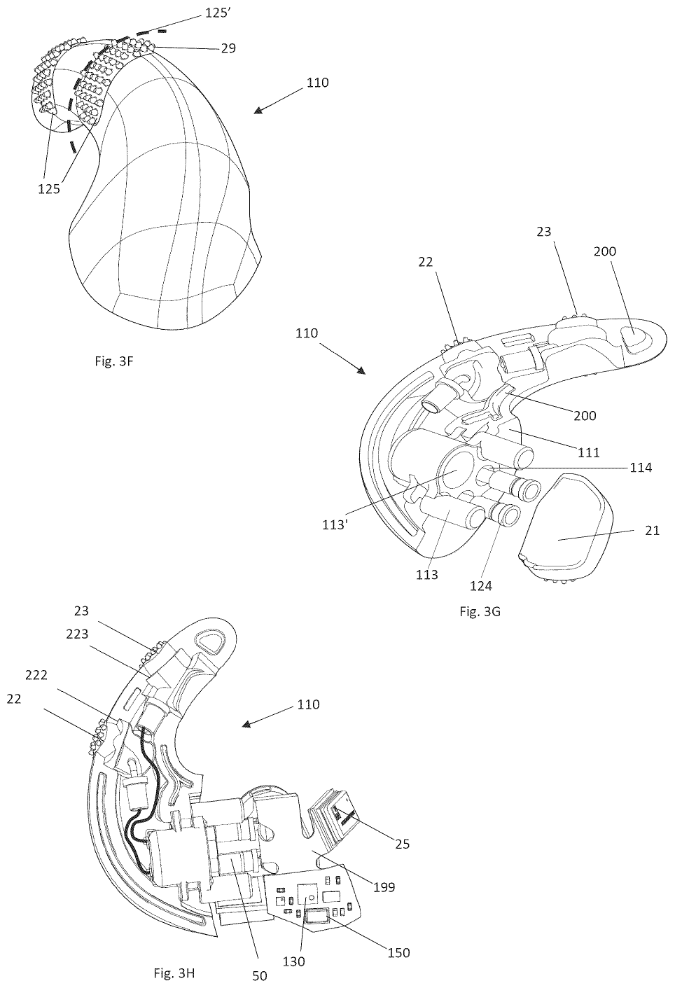

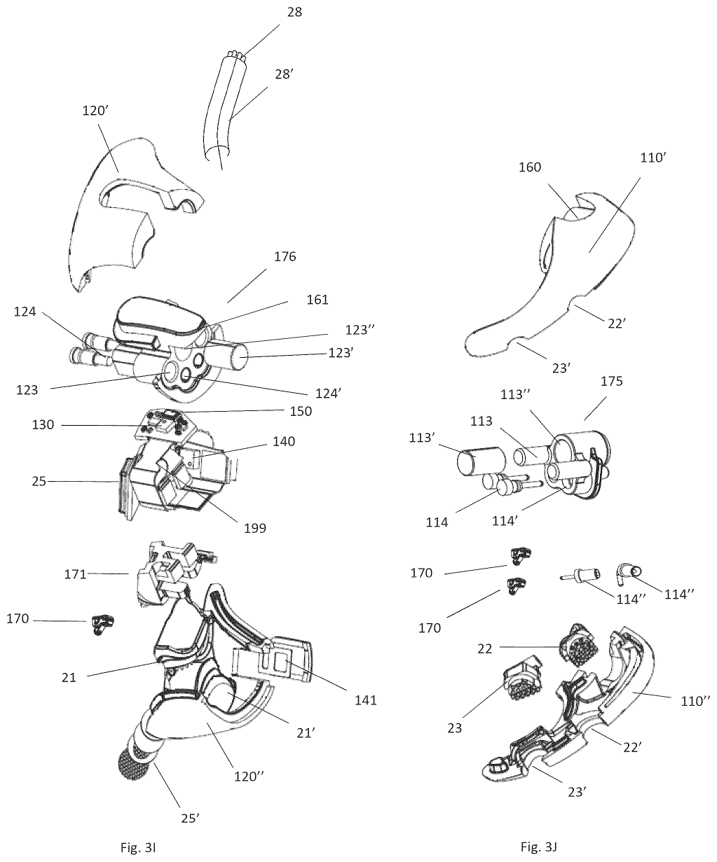

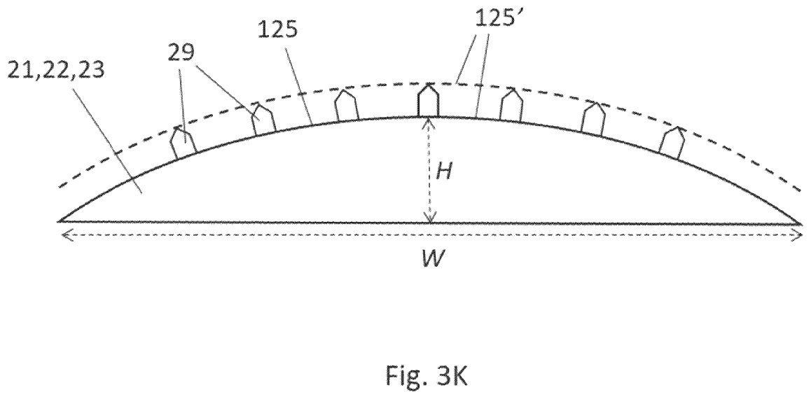

With reference to FIGS. 3A-3K, the first aspect of this disclosure provides a wearable electronic stimulating device 20 comprising: a distal portion 110 comprising one or more distal electrode modules 22,23, a proximal portion 120 comprising or being remotely connected to one or more of: a control module 130, and a power source 150 for powering the control module 130 and the distal electrode modules 22,23, the distal portion 110 having a cylindrical tapering towards the distal end resilient C-shape configured to fit inside the cymba concha of an ear 100 when the wearable electronic stimulating device 20 is in use, for providing contact towards the inside curve of the antihelix and the cymba concha of the ear, and each distal electrode module 22,23 having an outer curved surface adapted to, and arranged in, the outside surface of the distal portion 110 at a position for being brought in contact with the cymba concha of an ear when the wearable electronic stimulating device 20 is in use. It is also within the inventive concept of the first aspect to arrange any of the control module 130 and the power source 150, and any other components which are not physically required to be arranged in the wearable electronic stimulating device 20, in a standalone remote device 31, 32 being connected to the wearable electronic stimulating device 20 via an electrical interface contact 28. The electrical interface contact 28 may optionally be arranged on a mounted protruding extender 28′ to ensure easy access when in use.

In some embodiments, the proximal portion 120 and the distal portion 110 may be remote from each other and coupled by a resilient member biasing the proximal portion 120 and distal portion 110 away from each other. The resilient member may form a resilient C-shape. The resilient member is deformable to allow the proximal portion 120 and distal portion 110 to be mounted in the ear, for example with the proximal portion 120 positioned within the cavum concha and the distal portion positioned within the cymba concha. The bias force of the resilient member presses the proximal and distal portions pressed against the skin of the ear to ensure good sufficient between the electrode modules 21, 22, 23 and the skin. The material and geometry of the resilient member can be tailored to provide a specific bias force for optimum contact with the ear.

The electronic stimulating device 20 according to present disclosure provides a data driven non-invasive transcutaneous vagus nerve stimulation. The electrical pulse generating module may be controlled and driven by electronic circuits and/or firmware residing in the electrode control module 130 and/or optional remote devices 31, 32, such as a program residing in a smartphone 31, APP, or a remote computer 32, communicating with the electrode control module. The programs may use different impulses distributed through the electrode modules 21, 22, 23, the electrode modules being electrically connected 50 to the electrode control module 130, for relief of pain and/or stress, for better sleep, for less anxiety, or for athletic recovery etc. These impulse patterns may be regulated through individual unique pain relief programs in the smartphone 31 controlled app, a remote computer 32 or the electrode control module itself.

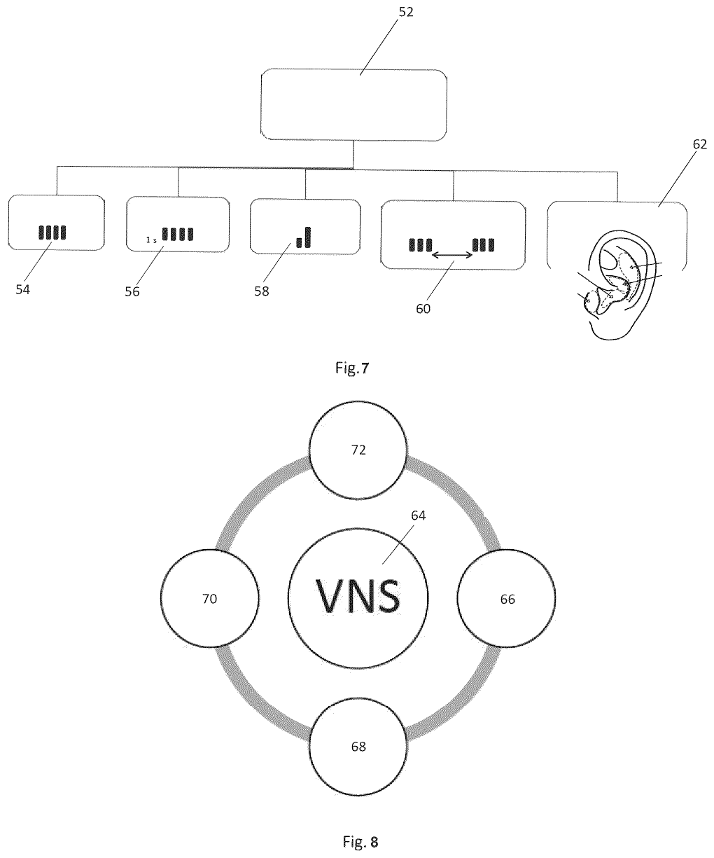

The wearable electronic stimulating device 20 according to present disclosure is provided as an in-ear-piece, advantageously formed to fit into the cymba conchae of an ear, as shown in FIG. 3B, but other forms may also be adapted. In one embodiment it is provided an electronic stimulating device 20 according to present disclosure using innovative carbon infused silicone which is able to drastically improve ear-fit and stimulation surfaces. In some embodiments the battery and all electronics, such as printed circuit board, components, battery, and wiring are contained in the wearable earpiece. In other embodiments, some of the components, such as battery and/or electrode control module may be comprised in a separate housing, for being carried separately from the ear piece(s) as illustrated in FIG. 6, and being electrically connected through a signal/power cable 6. The separate housing may for example be carried on the neck.

The electronic stimulating device 20 according to present disclosure is a t-VNS apparatus able to individually stimulate multiple auricular areas of the vagus nerve with precision, comfort, and high amplitude. Accuracy, strength, and comfort have been determined to be important conditions for alleviating chronic pain.

It was postulated that pain relief from the auricular branch of the vagus nerve can be achieved and amplified through using multiple sites of the ear. It was also postulated that these different sites have unique independent potential to create pain relief. It aligns with cadaver studies showing that the distribution, function, and thickness of myelinated axons along the auricular branch of the vagus nerve, differs greatly between individuals. An effective t-VNS apparatus was invented to target several points of the auricular branch of the vagus nerve, and do so directly, not as an artifact of impedance. Precision stimulation creates more action potentials because it can deliver a higher amplitude in the desired area. This was detected to be crucial, enabling activating a certain number of a-fibers is necessary for t-VNS to be effective, and these myelinated axons need high amplitude to be triggered.

According to the present invention, the electrode control module 130 may individually control the transmitted electrical pulses to the one or more electrode modules 21, 22, 23. Thus, when arranging the electrode modules 21, 22, 23 adjacent key positions in the ear, and thus targeting several points of the auricular branch of the vagus nerve 10 directly, substantially better efficiency for pain relief is achieved, and with better comfort and amplitude.

The electrode control module 130 controls and modify the transmitted electrical pulses in one or more of: duration: wavelength: pulse width: amplitude: frequency; and pattern.

As illustrated in FIG. 7 it is shown several possible variation parameters 52 for controlling the pattern of the electrical pulses used in the electronic stimulating device 20 to influence the efficacy of the stimulation. The signal itself, but also to where the signals are sent, which of the electrode modules 21, 22, 23 and dependent on where the individual electrode modules 21, 22, 23 are located.

Table 1, below, illustrates various electrical pulse schemes/programs that are enabled with the electronic stimulating device 20 according to the present disclosure, with the electrode modules 21, 22, 23 targeting different regions of a human ear.

Table 1 and FIG. 7 show that different electrical pulse programs can vary by any of the following parameters: target ear area 62: pulse width (μs) 56: pulse frequency (Hz): pulse amplitude (mA) 58; duration of stimulation (“stim on”; s); duration of no stimulation (“stim off”; s) 60; total time duration of the program (mins); time of day for applying the program (morning/evening); frequency of applying the program (days per week); and the treatment time span (number of weeks). The combination of the “stim on” and “stim off” durations with the pulse width, frequency and amplitude enable precisely tailored pulse patterns to be generated, for example with more complexity than previously considered. The number of stimuli/pulses 54 may be another parameter considered, for example as a combination of pulse frequency and duration of stimulation.

By using these parameters to tailor the operation of the electronic stimulating device 20 to deliver stimulation to certain areas of the ear, different treatment effects can be achieved. As shown in the first column of Table 1, the different programs can be tailored to have different effects on a user. For example, for pain relief treatment the electronic stimulation device 20 may be operated according to any of treatment programs 2, 3 and 6.

TABLE 1
Examples of various electrical pulse schemes

| | | Pulse | | | Duration | Duration | Total | | | | |
|---|---|---|---|---|---|---|---|---|---|---|---|
| Treatment | Area targeted | width | | | stim | stim | time | | | | Time |
| program | by electrode | (μs) | Hz | mA | on (s) | off (s) | (mins) | Morning | Evening | Frequency | span |
| 1 (Sleep) | Inner tragus | 20 | 8 | low | 30 | 60 | 30 | | x | 5 out of 7 | 2 weeks |
| | (anterior) | | | | | | | | | days | |
| 2 (Pain) | Cymba conchae | 250 | 25 | To tolerance | 420 | 660 | 60 | x | | 5 out of 7 | 6 weeks |
| | | | | (max 8) | | | | | | days | |
| 3 (Pain) | Inner tragus | 500 | 25 | To tolerance | 360 | 420 | 60 | x | | 5 out of 7 | 6 weeks |
| | (anterior) + | | | (max 8) | | | | | | days | |
| | Cymba conchae | | | | | | | | | | |
| 4 (Restitution) | Inner tragus | 500 | 25 | low | 60 | 60 | 60 | | x | 5 out of 7 | 4 weeks |
| | (anterior) | | | | | | | | | days | |
| 5 (Depression | Auricular | 1 | 20 | 4 to 6 | constant | constant | 30 | | x | 5 out of 7 | 4 weeks |
| and anxiety) | conchae | | | | | | | | | days | |
| 6 (Pain) | Inner tragus | 250 | 25 | To tolerance | 360 | 90 | 30 | | x | 5 out of 7 | 6 weeks |
| | (anterior) + | | | (max 8) | | | | | | days | |
| | Auricular | | | | | | | | | | |
| | conchae | | | | | | | | | | |
| 7 (Restitution) | Auricular | 250 | 25 | low | 30 | 60 | 30 | | x | 5 out of 7 | 4 weeks |
| | conchae | | | | | | | | | days | |

According to the present disclosure the number of electrode modules 21, 22, 23 are two or more. In the embodiment shown in FIG. 3A, the electronic stimulating device 20 comprises three electrode modules 21, 22, 23.

The electronic stimulating device 20 may be adaptable to be arranged at the ear concha and tragus and/or the antihelical fold and conchal bowl of an ear. Since two or more electrode modules 21, 22, 23 are advantageously comprised in the electronic stimulating device 20 of the electronic stimulating device 20, there has been provided a wearable device designed to cover multiple areas that is to be targeted, for example as highlighted in Table 1 above. Electrode modules 21, 22, 23 may be paired to triangulate the electrical stimulation signals within specific regions of the ear to provide stimulation to the target areas, for example those listed in Table 1.

FIG. 3B illustrates how the wearable electronic stimulating device 20 is worn in the ear, showing individual arrangement of the endpoints of the electrode modules 21, 22, 23. With additional reference to FIG. 2, it can be seen that the electrode modules 21, 22, 23 are arranged adjacent respective sub-branches 11,12,13,14 of the auricular branch of the vagus nerve 10. It is further postulated that, since the nerves present in the ear also comprise several further major nerves, the electronic stimulating device 20 may advantageously also be used to stimulate one or more of these, and that this will have positive effect on the pain relief effect.

In a first embodiment according to present disclosure, as shown in FIG. 3A, the proximal portion 120 of the electronic stimulating device 20 comprises one or more proximal electrode modules 21, the proximal electrode module 21 being controlled by the control module 130. The one or more proximal electrode modules 21 have an outer curved surface 125 substantially adapted to, and arranged in, the outside surface of the proximal portion 120 at a position for being brought in contact with the cavum concha of an ear when the wearable electronic stimulating device 20 is in use. The curvature may deviate from the curvature of the proximal portion 120. Deviation may arise from changed size of the electronic stimulating device 20 and the need to standardize on one or a few size electrode modules. The curvature need not be circular but may in one embodiment be a central flatter portion and a rounded design in the edge area of the electrode module 21.

In some examples, the C-shaped distal portion 110 may have a cylindrical tapering form that is formed with an outside curved form intended for maximizing the contact with the cymba concha of a user's ear.

Any of the electrode modules 22,23,21 are electrically paired with another of the electrode modules 22,23,21, and the control module 130 is configured to output a controlled voltage and/or current signal over the one or more paired electrode modules 22,23,21. Normally only two electrode modules 21, 22, 23, which are paired, are active at the same time. An output pattern and pairing matrix may be dynamically changed in the time domain within the same output sequence. For example, for a period of 1 second driving current between the two electrode modules 22, 23 on the distal portion, and the next 2 seconds driving a current between the inner electrode module on the distal portion and the electrode module 21 on the proximal portion, so on. The output signal is configured to output a constant current value as defined. In practice the impedance may vary, at least between users, and the voltage is thereby dynamically altered to achieve a constant or desired current flow between the paired electrode modules. When impedance changes during an output session, due to mechanical pressure on the electronic stimulating device 20, or changed humidity, temperature or the like, the voltage is then altered to maintain current output via the electrode modules.

The control module 130 is configured to control the pairing configuration of the electrode modules 22,23,21. The pairing configuration may be preconfigured, remotely or locally controlled, or dynamically adjusted as a response to input sensed by a sensor 27, 30, 140.

The control module 130 may be configured to output a predefined current over the electrodes 22,23,21 at a predefined pattern and polarity over a predefined pairing scheme of the electrodes 22,23,21.

For monitoring user response (i.e., physiological feedback) and status it may be advantageous to provide a heart rate sensor 140 in the electronic stimulating device 20 (shown in FIG. 3I). The heart rate sensor 140 may be a Photoplethysmography, PPG, sensor, where in one embodiment the sensor 140 is arranged behind a sensor window 141 arranged in the proximal portion 120 of the electronic stimulating device 20, as shown in FIG. 3A. Other types of sensors are possible, with or without a protective glass/window 141. The heart rate sensor 140 may typically be able to monitor heart rate, heart rate variability (HRV) beat composition, blood pressure and other.

With reference to FIGS. 3A, 3E and 3K, for improved current output efficiency the outer surface 125 of one or more of the electrode modules 22,23,21 comprises an array of multiple spikes 29. The surface of skin is composed of a hard layer of non-conducting dead cells. This outer, protective layer is referred to as the epidermal stratum corneum. It has been seen that a better transmission of electrical pulses between the electrode modules 21, 22, 23 and the area wherein the various areas of the auricular branch of the vagus nerve 10 that are targeted is located is achieved if the contact points of the electrode modules 21, 22, 23 are provided with spikes for a better electrical contact with the ear.

The spikes 29 may be designed with a height h between 0.1 and 0.9 mm, and more advantageous between 0.2 and 0.6 mm, and most advantageous between 0.3 and 0.5 mm. The size and number are adaptable to meet the requirement of the use case. It is proposed that for use cases wherein the ear of a user is large, typically in large live stock or other animal cases the sizes and number of spikes in the electrode module is increased. Likewise, it may be reduced for smaller sized users.

The following numbers and sizes are typically adapted to the form and size of a human ear. But, even in these cases it may be envisaged to alter the size recommendations.

The spikes 29 may be arranged with at least 0.2 mm distance w between spikes, or more advantageous with at least 0.4 mm distance between spikes, or most advantageous more than 0.6 mm distance between spikes.

In some embodiments, the outer surface 125 of the electrode module 21, 22, 23 has a convex curve and the spikes 29 are provided across the convex surface, as demonstrated in FIGS. 3E, 3F and 3K. The convex curve of the outer surface 125 of each electrode module 21, 22, 23 may not match the curve of the outer surface of the first portion 110. The curve of the electrode module outer surface 125 may be designed for optimal contact with and penetration of the skin when the device 20 is worn in the ear.

The convex outer curved surface 125 may follow the curve of a low circular-arc profile 125′ having gentle curvature and a smooth rounded apex. This ensures shallow, controlled skin penetration specifically targeting the epidermal stratum corneum within the cymba concha, closely matching its anatomical contours.

The low convex circular-arc profile 125′ may be provided by the convex outer surface 125 having a chord width/length W and an arc height H in specific ranges, and/or a ratio W:H in a specific range. For example, W may be 14-21 mm, 16-19 mm and H may be 2-3 mm or 2.2-2.8 mm. Additionally or alternatively, the profile 125′ of the curved outer surface 125 may be given a radius of curvature R within a specific range. For example, R may be 10-25 mm or 14-20 mm.

The spikes 29 in the array may have a substantially constant height, so that the tips of the spikes 29 also follow the convex arc profile 125′ of the outer curved surface 125.

The spikes 29 may have a conical form, tapering towards the top, wherein the cone top of the spikes 29 is configured to be able to bend into or penetrate the epidermal stratum corneum (the outer, protective layer) of the skin of a user's ear.

The number of spikes 29 in any of the distal electrodes 22,23 are advantageously more than 20, or more advantageous more than 30, or most advantageous more than 40.

The number of spikes in any of the proximal electrodes 21 are advantageously more than 40, or more advantageous more than 60, or most advantageous more than 80.

The actual form, size and number of the spikes adopted in any embodiment according to present disclosure may vary and deviate from the numbers mentioned and the FIGS. without deviating from the inventive concept.

The electronic stimulating device 20 may comprise one or more sensors 27, 30, 140, being any one or more of: temperature sensor, magnetic and/or electric field sensor, humidity sensor, pressure sensor, accelerometer and gyroscope sensor, electrodermal activity and heart rate sensor.

One or more sensors for detecting a physiological signal responsive to a transcutaneous vagus nerve stimulation is provided, and the electrode control module and the one or more sensors 27, 30, 140, 146 being in communication via wired 28 or wireless communication channel 40 for transmitting sensor data from the one or more sensors 27, 30, 140, 146 to the electrode control module.

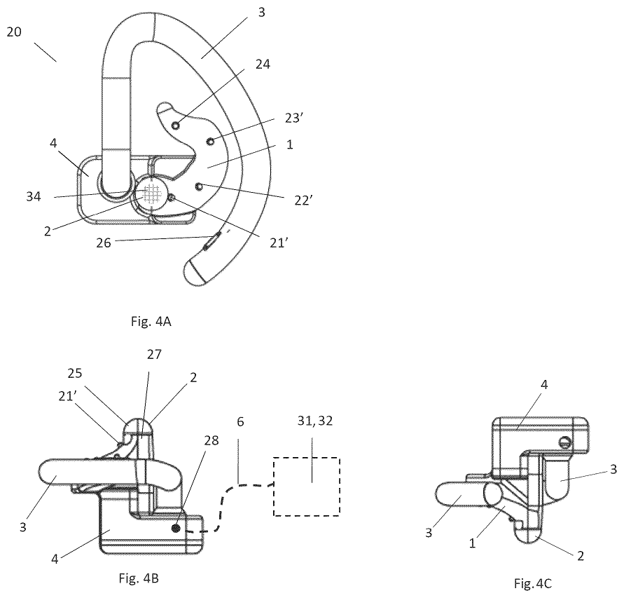

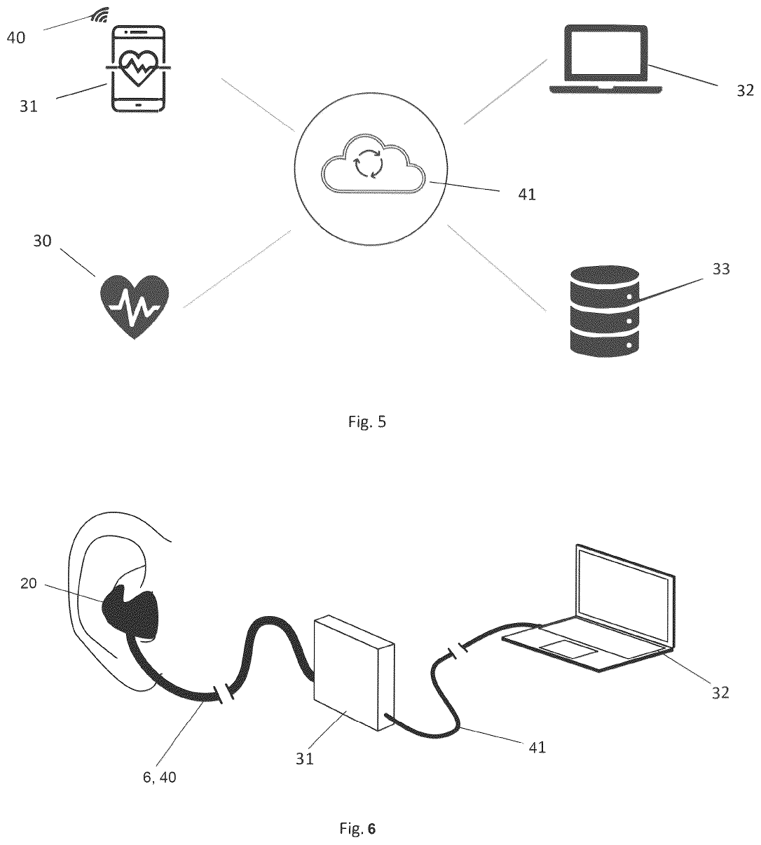

The one or more sensors 27, 30, 140, 146 may for example be arranged in the proximal portion 120 or the extension protrusion 2 (see FIGS. 4A-C) of the electronic stimulating device 20. In another embodiment the sensor may be provided by an, optionally further, standalone senor device 30, such as for example a Fingertip Pulse Oximeter, a wearable measuring health metrics like SpO₂, skin temperature, heartbeat, or the like, as indicated in FIG. 5.

The electronic stimulating device 20 according to present disclosure may stimulate reduction of the production of adrenaline, and increasing the level of cortisol and hormones that naturally regulate stress, blood pressure and inflammation. Both adrenaline level, cortisol level, and activity in the vagus nerve itself, are key influences in chronic pain.

The reduction in Heart Rate Variability, HRV, can be seen as a marker of health and main body functions, and this may increase stress resilience, optimize recovery, and make a person feel better and be more productive.

One or more sensors 27, 30, 140, 146 such as a heart rate monitor or an in-ear heart rate monitor may be provided for sensing how a person react to the stimulus from the electronic stimulating device 20. Other sensor data may be collected by the one or more other sensor types. The sensor data constitutes user data that may be used for optimizing the use of the electronic stimulating device and/or communicated via a user interface. The sensor data may further be used to generate and maintain Big Data Models, which again may be used for controlling the executing parameters of the electronic stimulating device 20. In one embodiment it is foreseen that the Big Data Models are used to train an artificial intelligence, AI, module for autonomous adjustment of the controlling parameters of the electronic stimulating device 20. Such AI controlling modules may be implemented in the electrode control module, or the remote processing device such as a smart phone 31 app or a cloud based server 32.

The one or more sensors 27, 30, 140, 146 may be one or more of, but not limited to: a pulse-oximeter: a sensor for electrodermal changes; and a sensor for electrical impedance.

Thus, the sensor data is not limited to HRV reaction data, but may be data retrieved from sensors measuring any measurable response from a person, such as, but not limited to: sweat, eye pupil change, muscle activity or other. The sensors may be a combination of different sensor types.

The control module 130 may further be configured to use input from the sensors 27, 30, 140 to set the output voltage level over the electrode module 22,23,21 pairs.

As shown in FIGS. 3C, 3D, 3E and 3G, the distal portion 110 of the electronic stimulating device 20 is a replaceable portion comprising a distal connector device 113,113′ and electrical connectors 114 for quick lock/release from the proximal portion 120, and the proximal portion 120 comprises a corresponding proximal connector device 123,123′ and electrical connectors 124 for cooperating with the distal connector device 113,113′ and electrical connectors 114.

The electrode modules 22, 23 are connected via connector plugs 114″ and electrical wiring 115 to corresponding electrical connector 114, and when the distal portion 110 is connected with the proximal portion 120 the electrode modules 22, 23 is further electrically connected via the electrical connector 124 in the proximal portion to the controller 130.

FIG. 3A shows the contact boundary 111, 121 of the proximal and distal portions 110, 120. FIGS. 3D and 3E further show an optional additional alignment feature wherein a protruding member 161 in the proximal portion 120 is provided for being inserted in the receiving recess 160 in the distal portion 110.

In an alternative embodiment, the electrical connectors 114,124 also comprise inbuilt both the connector features of the distal and proximal connector device 113,113′, 123,123′.

The connector devices 113,113′, 123,123′ may further comprise one or more of: a magnet 113′, 123′, a spring-loaded snaplock, a bayonet type connector, a threaded type connector, male/female alignment pin 113 and recess 123, and Registered jack, RJ for enhancing the connecting and holding ability of the connector device 113,113′, 123,123′.

It is also within the inventive concept to provide the wearable electronic stimulating device 20 with the proximal and distal portion integrated in one portion without the feature enabling quick separation of the portions.

FIGS. 31 and 3J show exploded views of the main components of respectively the distal and proximal portion 110,120.

A proximal connector module 176 comprising: a magnet recess 123″ for receiving the magnet 123′, two female alignment pin recess 123, and two electrical connector pin recess 124′ for receiving the two electrical connector pins 124. The electrical connector pin 124 is typically comprising some form of spring-loaded feature ensuring that there is always a certain contact pressure to a receiving contact point in the distal connector module's electrical connector 114 contact surface.

A distal connector module 175 comprising: a magnet recess 113″ for receiving the magnet 113′, two male alignment pins 113, and two electrical connector contact surface device recess 114′ for receiving the two electrical connector 114 contact surface devices.

When the distal and proximal portions 110,120 as shown in FIGS. 3E and 3D are assembled and connected to each other as shown in FIG. 3A, the male/female alignment pin 113 and recess 123 ensure correct alignment of the two parts, and the magnets 113′, 123′ maintain the connection once connected together. The electrical connectors 114,124 make contact due to the spring-loaded feature of the electrical connector pins 124.

In order to enhance the features of the electronic stimulating device 20 the proximal portion further comprises a speaker device 25 arranged for outputting sound, the speaker device 25 being electrically connected to and controlled by the control module 130. The speaker resides behind a membrane assembly 25′ configured to pass on sound waves to the inner ear of a user. The speaker device 25 is arranged on a proximal print card chassis 199 arranged in the inner space of the proximal portion 120.

The electronic stimulating device 20 may comprise a communication device for enabling communication between the electrode control module 130 and a remote control module.

The communication device may further provide communication between two control modules 130 arranged to be used by a same user. Thus the signal pattern may be controlled in the two devices for optimal effect.

The electrodes modules 22,23,21 and the distal and proximal portion 110,120 may provide dust proof, splash proof and/or water resistant enclosures to protect electronic components within. They may advantageously provide waterproof enclosures, wherein the electronic components inside the distal and proximal portions 110,120 are connected in a dry environment. In one embodiment the electrodes 21, 22, 23 are mounted in conduits 21′, 22′, 23′ provided in the distal/proximal portion wall such that a base 171 (not shown for the distal portion 110) inside the distal/proximal portion provides supporting base for a spring-loaded electrode holding module 170 to press the electrode module towards an electrode seat-recess 222, 223 in the distal/proximal portion wall in order to maintain good protection against water ingress.

The production and assembly of the parts of the proximal and distal portion 120, 110 can be done in multiple ways. One alternative is to produce housing portions for each of the proximal and distal portions 120, 110 in two halves 120′, 120″, 110′, 110″, then a first half of each portion may have protruding elements 200 (see FIG. 3G) being paired with corresponding recesses (not shown) in the second half and then being pressed together after assembly and mounting of the components in the portion.

FIG. 3H shows the components arranged in a first half of the distal portion 110, together with some of the components that are to be arranged in the proximal portion 120. The FIG. does not show any of the proximal portion housing halves.

Each of the proximal and distal portions 120, 110 may be coated with a durable and resilient layer of silicon or the like on area exposed to the environment and which does not cover the sensors or electrode modules.

The housing of the proximal and distal portion 120, 110 may advantageously be produced by a material having a certain resilience, such that when fitting it to an individual ear it forms and provides a spring-loaded effect to hold the device 20 in place. The material may be a flexible material, able to change form by applying pressure, moderate heat, or other. With reference to FIGS. 5 and 6, in a further embodiment the electronic stimulating device 20 according to present disclosure comprises:

a remote processing device 31, 32 comprising communication modules for communicating with the electrode control module 130, the remote processing device further comprising one or more of:
an application, app, for communicating one or more of:
   electrode control module operating mode,
   sensor data, and
   operating mode selection,
storage 33 for storing data transmitted from the electrode control module,
communication module 41 for communicating data to/from a server/cloud computer 32.

In one embodiment the remote processing device is a smart phone 31.

It may be advantageous to provide an app that is executable on a smart phone for the convenience of keeping the cost level down. It is also generally accepted to use a smart phone for applications related to sensors and health improving gadgets. A smart phone provides easy communication features both for communicating between the app and the electronic stimulating device 20 according to present disclosure, and any sensors or modules associated with the electronic stimulating device 20. It is further advantageous to use the communication protocols supported by the smartphone to provide a communication channel between the electronic stimulating device 20 and any remote processing services such as for example provided as a cloud service. The smart phone may be substituted with any other processing device comprising communication features and components associated with executing special purpose programs provided for the operation of the electronic stimulating device 20.

The electrode control module may further control the electrical pulses transmitted to each individual electrode module 21, 22, 23 based on feedback from the one or more sensors 27, 30, 140. It is thus possible to monitor HRV reactions to any changes made to the characteristics of the electrical pulses. One of the draw backs of any of the prior art techniques is that any current above the 1-2 mA region is becoming extremely uncomfortable or painful for a person using one of those supported devices. Using the electronic stimulating device 20 according to present disclosure is possible to stimulate multiple areas, for example two or more locations of the auricular branch of vagus in sequence using for example a 0-500 ms pulse width of 8-30 Hz and current strength in the region of 0-8 mA, or higher. This is used as an example only, but the embodiments shall comprise a sequence comprising any pair combination of any of the electrode modules 21, 22, 23, and also a sequence not depending on the order of appearance. Also for some sequence schemes may exclude the use of one or more of the available electrode modules. Alternative embodiments of the device of present disclosure may comprise more electrode modules than illustrated in the figures. Being able to alter the stimulation changing between several stimulation areas in sequence may enable a higher electrical power/current to be distributed less side effects/discomfort.

In a further embodiment of the electronic stimulating device 20 according to present disclosure each electrode module may individually stimulate any of several auricular branches of the vagus nerve, and several auricular branches of the vagus nerve may simultaneously be stimulated. Although many of the embodiments discussed in present disclosure mentions the auricular vagus nerve, it should be understood that any of the major nerve types being present in an ear may be stimulated in the same manner, in combination with stimulation of the auricular vagus nerve, or separate. This includes, but is not limited to: the auriculotemporal nerve 15, the great auricular nerve 16, and/or other cranial nerves.

In a further advantageous embodiment of the electronic stimulating device 20 according to present disclosure the number of electrode modules are three or more. It is thus possible to stimulate at least three individual and separate branches of the auricular vagus nerve in sequence as discussed above.

It has been proven that a precise, intelligent, multisite stimulator may increase efficiency. The electronic stimulating device 20 according to present disclosure may stimulate three or more densely myelinated sites along the auricular branch of the vagus nerve, ABVN, and have the capability to do so independent of one another. As the necessary density of axons for vagal activation has huge intra-individual variability, the electronic stimulating device 20 according to present disclosure may stimulate different sites while tracking changes in HRV in real-time, optimizing vagal activity. The HRV data will form the basis for provided machine learning software, which may create clinical stimulation parameters for use in the electronic stimulating device 20 according to present disclosure.

In a second embodiment of the electronic stimulating device 20 according to present disclosure, as shown in FIG. 4A-C, the electronic stimulating device 20 comprises a control module 4 and one or more heat emitting areas 34. The electrode control module 4 may further comprise an extension protrusion 2 for being inserted into an ear canal, and at least one of the heat emitting areas 34 may be provided on the extension protrusion 2.

The electronic stimulating device 20 comprises base entity 1 adaptable to be arranged at the ear concha and tragus and/or the antihelical fold and conchal bowl of an ear. Two or more electrode pins 21′, 22′, 23′, 24, 26 are provided in the base entity of the electronic stimulating device 20. This provides a wearable device designed to cover multiple areas of the ear that are to be targeted with stimulation pulses, for example as highlighted in table 1 above. FIGS. 4A-4C illustrate the wearable device, showing individual arrangement of the endpoints of the electrode pins 21′, 22′, 23′, 24, 26. With additional reference to FIG. 2, it can be seen that the electrode pins 21′, 22′, 23′, 24, 26 correspond to being arranged adjacent respective sub-branches 11,12,13,14 of the auricular branch of the vagus nerve 10. It is further postulated that, since the nerves present in the ear also comprise several further major nerves, the electronic stimulating device 20 may advantageously also be used to stimulate one or more of these, and that this will have positive effect on the pain relief effect.

In one embodiment according to present disclosure the electronic stimulating device 20 comprises an earhook 3 as seen in FIG. 4A-4C for being arranged over and around the base of an ear. Thereby a better, safer and stable arrangement of the electrode pins 21′, 22′, 23′, 24, 26 may be achieved than with just the base entity 1.

In yet another embodiment, one more of the electrode pins 21′, 22′, 23′, 24, 26 may be arranged in the earhook 3. Thus, electric stimulation may be distributed towards nerves from the backside of the auricle of the ear.

One or more of the electrode pins 21′, 22′, 23′, 24 comprised in the base entity 1 is advantageously designed with a base portion and a protruding portion as exemplified in FIG. 4B, such that the protruding portion is designed to exactly target an area of a contacting ear corresponding to a specific underlying nerve junction, such as a specific branch of the ABVN or other nerve. The pin width pw of the upper portion of the electrode pins 21′, 22′, 23′, 24, 26 may be narrow; as small as less than 5 mm, and even more advantageously less than or equal to 2 mm.

The one or more of the electrode pins 21′, 22′, 23′, 24, 26 arranged in the earhook 3 may be designed with a broader contact surface, for example about 5-10 mm².

The material used in the base entity 1 and/or the earhook 3 of the electronic stimulating device 20 may advantageously be produced by a material being flexible and formable, such that when fitting it to an individual ear it takes on and holds a form until actively being changed to another form. The material may be a flexible material, able to change form by applying pressure, heat, or other.

The electronic stimulating device 20 according to the second embodiment comprising an in-ear piece 1, 2 and advantageously comprising a further holding element 3 for reaching around and to the backside of the ear, the further holding element 3 alternatively comprising electrode pins 21′, 22′, 23′, 24, 26 for arrangement near the antihelix of an ear.

The heat emitting area 34 is shown as an integrated portion of the electronic stimulating device 20 according to the second embodiment, preferably into the extension protrusion 2 for being inserted into an ear canal, as seen in FIGS. 4A, 4B and 4C. This is for illustration purpose only, and it should be understood that the heat emitting area(s) 34 may also be comprised in any other area of the electronic stimulating device 20 according to the second embodiment. Typically the heat emitting area will be constructed as a heat emitting electric wire coupled to the power source, and controlled by for example the electrode control module. Any other form and construction of a controllable heat emitting area/device may be comprised, such as a standalone heat emitting device (not shown) being controlled by the electrode control module or other remote device such as a smart phone app. The actual form, size, construction, and number of the heat emitting areas adopted in any embodiment according to present disclosure may vary and deviate from the FIG. without deviating from the inventive concept.

The heat emitting areas 34 may be controlled according to a predefined heat scheme and/or feedback from the sensors 27, 30.

Using the heat in combination with electrical stimulation via the electrode pins 21′, 22′, 23′, 24, 26 is postulated having a soothing effect, and thus larger currents may be used in the electrode pins 21′, 22′, 23′, 24, 26 without increasing the discomfort of use to the user. The heat is further believed to prevent or at least reduce discomfort and improve responsiveness of the stimulation. For example if the user is a comatose person, the heating feature may be used to ensure the user is react with less discomfort and thus may be more responsive to the electrical signals distributed by the electrode pins 21′, 22′, 23′, 24, 26.

The electronic stimulating device 20 according to the second embodiment may stimulate each one of one or more of the electrode pins 21′, 22′, 23′, 24, 26 with a current strength up to 10 mA, and may use the built-in resistance in the extension protrusion 2 or the electronic stimulating device 20 to create heat emitting areas 34. The heat is applied to the receiving areas close to the electrode pins 21′, 22′, 23′, 24, 26 for concealing unpleasant sensations related to the high amplitude stimulation and make the stimulation more pleasant and feasible.

In a related use scenario the electronic stimulating device 20 according to the second embodiment may be used in clinical trials, wherein the heat emitting areas 34 can function as a sham stimulation, solving a problem for scientific communities, wherein it may be difficult to camouflage a scam device in operation. Thus the electronic stimulating device 20 according to the second embodiment may be used as a scam device only delivering heat and not electronic stimulating pulses, to fulfill the requirement of test scenarios wherein a certain percentage of test personnel shall be objects to scam process to verify the efficiency of the correctly operating devices and methods.

It should be understood that the features and characteristics of the various embodiments presented in present disclosure can be adapted for use in combination with any of the other embodiments described. For example the heat emitting area 34 of the second embodiment may be implemented and used for similar purpose in the first embodiment, and the audio output of the first embodiment may be used for similar purpose in the second embodiment. It should further be understood that features of the electrode modules 21, 22, 23 of the first embodiment may be substituted or supplemented by the electrode pins 21′, 22′, 23′, 24, 26 of the second embodiment and vice versa. The same goes for the holding element 3 of the second embodiment and the distal portion 110 of the first embodiment. These examples do not exclude that other parts and features are used across the embodiments described.

The second aspect of this disclosure shows a method for operating a wearable electronic stimulating device 20, comprising the steps: providing one or more wearable electronic stimulating devices 20 according to the first aspect, arranging the one or more electronic stimulating devices 20 according to present disclosure in the cymba concha and cavum concha of a user's ear, where one or more electrode modules 21, 22, 23 of the electronic stimulating device 20 can stimulate several auricular branches of the vagus nerve in the ear, and further providing a current output pattern for the control module 130, arranging for the wearable electronic stimulating device 20 to output a voltage/current according to a provided output pattern.

The method may further comprise the following steps:
arranging one or more sensors 27, 30, 140, 146 for detecting a physiological signal responsive to a transcutaneous vagus nerve stimulation, and
adjusting one or more of the duration, wavelength, pulse width, amplitude and frequency of the electrical pulse to the one or more electrode modules 21, 22, 23 in accordance with the detected physiological signal responsive to the transcutaneous vagus nerve stimulation.

The method may further comprise, when the number of electrode modules 21, 22, 23 are paired:
transmitting electrical pulses to the paired electrode modules 21, 22, 23 in accordance with a configurable transmitting sequence.

Although FIGS. 3A-3J show two electrode modules in the distal portion it should be understood that the number of electrode modules may be more than two such that more areas in the cymba concha can be targeted. The same is true for the number of electrode modules in the proximal portion which may be more than one such that more areas in the cavum concha can be targeted.

In one embodiment of the invention only two of the electrode modules may be paired for outputting a signal at a time. This means that only one pair of electrode modules are active at any point in time. During a time sequence of using the wearable electronic stimulating device pairing may change to optimize output and coverage of several target areas.

The method may further comprise providing one or more heat emitting areas 34, and activate heat radiation from the heat emitting areas.

The method may further comprise adjusting the heat emitted from the heat emitting areas 34 in response to the detected physiological signals responsive to the transcutaneous vagus nerve stimulation.

The method may further comprise administrating activities, such as using the audio output 25 in response to the detected physiological signal responsive to the transcutaneous vagus nerve stimulation, the external activities being one or more of playing a tune or sound, instructions for meditation, and instructions for breathing techniques.

The third aspect of this disclosure shows a system for operating a wearable electronic stimulating device comprising: one or more wearable electronic stimulating device according to any of the first aspect, and a remote control module for controlling the one or more wearable electronic stimulating device 20.

It may further be provided one or more communication networks 40, 41 for providing communication channels between the electronic stimulating devices 20 and the remote processing device 31, 32.

It may be advantageous to use wearable electronic stimulating device 20 in a setup where two wearable electronic stimulating device 20 is provided and mounted to each of the left and right ear of a user respectively. The stimulating pattern and order may then alter between all the electrode modules of both electronic stimulating devices 20.

According to another aspect, there is provided a method for treatment of a user's chronic pain conditions and operating a wearable electronic stimulating device, comprising the steps: providing and arranging a wearable electronic vagus nerve stimulating, VNS, device, applying vagus nerve stimulation using the VNS device, and:
monitoring and recording the user's physical symptoms, and/or
providing to the user behaviour instructions, combining the vagus nerve stimulation with the monitored and recorded physical symptoms, and/or the behaviour instructions,
for generating a personalized treatment plan for managing the user's chronic pain.

The method offers a comprehensive, non-invasive approach to chronic pain management, leveraging the latest advancements in digital health technology and integrative therapies. The mobile application as discussed below ensures easy access and personalized support, making it a valuable tool for individuals seeking effective pain relief and improved quality of life.

Chronic pain conditions are prevalent and often difficult to manage effectively with conventional therapies. The treatment method may include physical therapy, medication, cognitive-behavioral therapy, or invasive procedures, which may bring limited relief or unwanted side effects. Integrative approaches that marry physical, psychological, and neuromodulation techniques hold promise for more effective management of chronic pain.

According to some embodiments, the VNS is a transcutaneous auricular VNS, taVNS, device arranged on a user's ear.

Vagus Nerve Stimulation, VNS, are available in many forms, which all are possible to be comprised in the novel treatment method proposed in the present disclosure. Further, a novel electronic stimulating device 20, is provided in present disclosure as described above, for enabling transcutaneous auricular vagus nerve stimulation. It has been seen that combining the taVNS of present disclosure with medication, cognitive-behavioral therapy, or invasive procedures has shown unparalleled results in treatment of chronic pain.

According to some embodiments, the vagus nerve stimulation is controlled by: providing a current output pattern for the control module, and arranging the wearable electronic stimulating device for outputting a voltage/current according to a provided output pattern.

In table 1 above it is shown typical embodiment of examples of various electrical pulse schemes, wherein low current is typically below 4 mA, medium current is in the interval 4-6 mA, and high current in the region 6-8 mA. Different levels may be defined for low, medium and high current, for example, but not limited to: low: 0-3, medium 3-7, high 7-10 mA, or low: 4-5, medium: 5-10, high: 10-15 mA, or low: 0-10, medium: 10-20, high: 20-30 mA. In rare cases the range may even be stretched up to as much as 100 mA with corresponding intervals.

The invention according to present disclosure relates to a method of treatment for pain management through the combination of pain reprocessing techniques such as interoceptive exposure (including somatic tracking and provocative testing), pain-relieving hypnosis based on advanced techniques for pain management, transcutaneous auricular vagus nerve stimulation, taVNS, heart rate variability, HRV, tracking, and various meditation practices, delivered through a dedicated platform. The platform may be defined by, but is not limited to: iOS and/or Android application.

According to some embodiments, the monitoring and recording physical symptoms the method comprising using interoceptive exposure where the user complete standardized movements, visualisations and actions to provoke symptoms in a controlled manner logging one or more of: pain intensity, frequency, and duration.

Interoceptive exposure such as somatic tracking and provocative testing is used to expose for painful movement in a safe way through visualization as well as monitoring and evaluating physical symptoms and pain triggers. Feedback from the tracking and testing are used as parameters for a feedback provided to the vagus nerve stimulation, and the control module controlling the signals provided by the VNS.

Interoceptive exposure is developed to build upon the principles of pain reprocessing and interoceptive awareness. The invention according to present disclosure provides mobile application including tools for users to log their physical symptoms, frequency, intensity, and duration of pain episodes using standardized scales, such as the Visual Analog Scale, VAS, or the Numeric Rating Scale, NRS. Interoceptive exposure further may involve a series of exercises of standardized visualizations movements and actions designed to provoke symptoms in a controlled and reproducible manner. This approach helps users reprocess their pain by confronting and understanding it in a structured way, and may comprise, but is not limited to:
each exercise may include detailed instructions, video demonstrations, and prompts for real-time symptom feedback;
advanced algorithms may be provided and be used to detect patterns and correlations in the user's pain data, cross-referencing this with contextual data such as activity level, mood, and environmental factors; and
visual analytics, such as trend graphs and heat maps, may further be provided to help users and clinicians identify pain triggers and understand their pain progression over time.

Further topics related to Interoceptive exposure is Core Principles and Modern Refinements. While pain reprocessing therapy, PRT, and related techniques share core principles with earlier work, they are expanded and refined according to present disclosure and may comprise, but is not limited to:
Cognitive Strategies: Unlike earlier focus on acknowledging emotional factors, PRT employs specific cognitive strategies to “retrain” the brain's pain response. These techniques actively alter pain pathways, moving beyond simply recognizing emotional connections to pain;
Emotional Awareness: Both approaches emphasize emotional awareness, but PRT and related therapies use more structured techniques to address emotional factors comprehensively. This structured approach facilitates deeper emotional insight and resolution for patients;
Structured Physical Activity: Similar to earlier processes, PRT encourages resuming normal activities. However, it adopts a more gradual and structured approach, ensuring patients build their confidence and physical capabilities progressively;
Neuroplasticity Focus: Modern methods emphasize actively rewiring neural pathways associated with pain to reinforce positive outcomes in pain reprocessing.

According to some embodiments, the monitoring and recording physical symptoms comprising monitoring a measure of heart rate variability, HRV, HRV defined as the root mean square of successive differences, RMSSD, between normal heartbeats, to track physiological responses and optimize treatment interventions, wherein the monitored HRV data is used to tailor one or more of hypnosis, meditation, and taVNS protocols to individual physiological states.

As seen in FIG. 8 it is visualized how the VNS device is central to the treatment of the patient, and how any of meditation 66, hypnosis 68, HRV 70, and movement 72 for provoking pain, may be used in combination with VNS for optimal pain treatment.

Pain-relieving hypnosis is given to the user to alleviate pain perception and improve relaxation. Reaction to such treatment is monitored, for example by a measured HRV, and may be used as input to the control module controlling the signals provided by the VNS.

Meditation practices may include Mindfulness-Based Stress Reduction, MBSR, pranayama breathing, and non-directive meditation to promote relaxation and enhance treatment efficacy. Reaction to such promoted relaxation is monitored, for example by a measured HRV, and may be used as input to the control module controlling the signals provided by the VNS.

Vagus nerve stimulation, VNS, such as the transcutaneous auricular vagus nerve stimulation, taVNS, of present disclosure may be used to modulate nerve activity and reduce pain.

The application supports and safely guides the use of a taVNS device, which can be worn on the ear or neck to deliver mild electrical stimulation to the auricular or cervical branch of the vagus nerve.

The application provides real-time instructions, feedback, and monitoring to ensure proper placement and use of the taVNS device in conjunction with interoceptive exposure exercises, hypnosis, and meditation practices.

Stimulation protocols are designed based on user-specific needs and validated clinical guidelines, including frequency, intensity, and duration of stimulation sessions.

This neuromodulation technique aims to reduce pain by modulating parasympathetic activity and reducing sympathetic overactivity.

According to some embodiments, the interoceptive exposure comprise somatic tracking and/or provocative testing.

A library of professionally developed hypnosis sessions targeting pain relief and relaxation based on scientifically validated and advanced pain management techniques is provided for use i combination with the VNS.

Based on a user's specific pain conditions, preferences, and needs, the user may select and customize sessions, including session length, background sounds, and focus areas.

The hypnosis sessions typically employ techniques including guided imagery, progressive muscle relaxation, deep breathing, and cognitive restructuring to alter pain perception and enhance relaxation.

Thus, according to some embodiments, providing to the user behaviour instructions comprise one or more of providing pain-relieving hypnosis sessions, and integrating meditation practices to provoke symptoms in a controlled manner.

Thus, as stated above, the integrating meditation practices comprise one or more of: Mindfulness-Based Stress Reduction, MBSR, techniques: pranayama breathing exercises: non-directive meditation; and personalized meditation plans,
to promote and/or enhancing one or more of: managing stress, relaxation, pain relief, safety, mental clarity, enhance treatment efficacy, and a comprehensive and integrative approach to pain management.

According to some embodiments, the method comprises: providing a dedicated computer platform, and configuring the computer platform by providing one or more applications running on the computer platform for providing one or more of: monitoring and recording the user's physical symptoms: controlling the wearable electronic vagus nerve stimulating device; and providing to the user behaviour instructions.

According to some embodiments, the computer platform comprise a mobile computer device. A mobile computer device is typically, but not limited to, a smart phone. Applications, dialogue and communication to back office/cloud based services may be provided by the computer platform.

According to some embodiments, the method comprises the steps:
integrating all treatment components within applications on the computer to execute one or more of: generating personalized treatment plans: employing algorithms to analyse interoceptive exposure data and dynamically adjusting hypnosis sessions: control the output of the deployed VNS device; and instructing on meditation practices; and
based on user-specific needs and ongoing feedback, providing one or more of real-time instructions, feedback, and progress tracking, to enhance treatment adherence and effectiveness.

According to some embodiments, the interoceptive exposure using one or more of:
standardized scales, wherein standardized scales being one of the Visual Analog Scale, or the Numeric Rating Scale, and
visual analytics such as trend graphs and heat maps to help users and clinicians identify pain triggers and understand pain progression.

According to some embodiments, the provocative testing component of the interoceptive exposure including one or more of detailed instructions, video demonstration, and prompts for real-time symptom feedback, to facilitate reproducibility of pain triggers.

According to some embodiments, the pain-relieving hypnosis sessions are based on pain management techniques the method comprises customized session parameters, the session parameters being one or more of duration, background sounds, and focus areas of the body or somatic topography, tailored to the user's specific pain conditions and preferences.

According to some embodiments, the method comprises the steps:
controlling the VNS device using stimulation protocols, wherein the stimulation protocols are based on one or more of clinical guidelines, with real-time monitoring, and individualized treatment needs by adjusting one or more of intensity, frequency, and duration, and
controlling the VNS device comprising providing a voltage/current output pattern for the control module, and arranging the VNS device on the user for outputting a voltage/current, according to a provided output pattern.

According to some embodiments, the method comprises: enhancing user engagement and adherence by one or more of push notifications, reminders, progress tracking, motivational support, and daily health tips to enhance user engagement and adherence.

Messages of these types may be given to user via a Graphical User Interface provided on the computer platform/mobile computer device by an instructor or by instructions given on any form.

According to some embodiments, the method comprises: providing secure cloud-based system comprising the computer platform, and the secure cloud-based system ensures one or more of data privacy, facilitating regular updates, and allowing for remote monitoring and telehealth integration by healthcare providers.

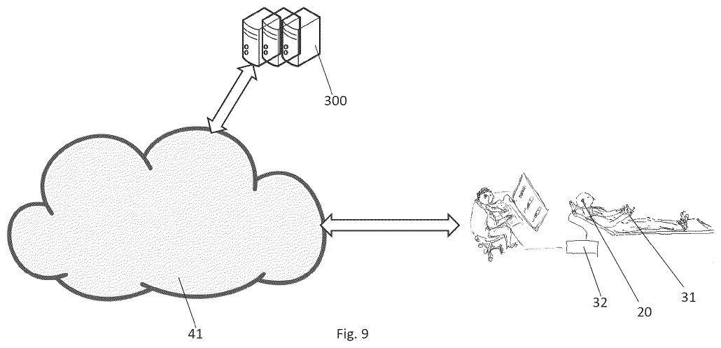

Providing for secure cloud based services as seen exemplified in FIG. 9 shows a scenario wherein the user 20 receives treatment according to present disclosure, a remote computer 32 and/or device 31, which may be operated by either the user or an optional on site therapist, is used for monitoring, controlling and guiding as discussed above. The computer 32 and/or device 31 may in a further embodiment be connected to a backoffice/cloud 41 service 300.

The person skilled in the art realizes that the present disclosure is not limited to the preferred embodiments described above. The person skilled in the art further realizes that modifications and variations are possible within the scope of the appended claims.

Additionally, variations to the disclosed embodiments can be understood and effected by the skilled person in practicing the claimed disclosure, from a study of the drawings, the disclosure, and the appended claims.

What is claimed is:

1. A method for treatment of a user's chronic pain conditions and operating a wearable electronic stimulating device, comprising:
providing and arranging a wearable electronic vagus nerve stimulating (VNS) device;
applying vagus nerve stimulation using the VNS device;
monitoring and recording the user's physical symptoms;
providing to the user behavior instructions; and
combining the vagus nerve stimulation with the monitored and recorded physical symptoms, and/or the behavior instructions, for generating a personalized treatment plan for managing the user's chronic pain.

2. The method of claim 1, wherein the VNS is a transcutaneous auricular VNS (taVNS) device arranged on a user's ear.

3. The method of claim 1, wherein providing the vagus nerve stimulation comprises:
providing a current output pattern for a control module; and
arranging the wearable electronic stimulating device for outputting a voltage/current according to the provided current output pattern.

4. The method of claim 1, wherein the monitoring and recording physical symptoms comprises using interoceptive exposure where the user completes standardized movements, visualizations, and actions to provoke symptoms in a controlled manner, and wherein monitoring and recording the user's physical symptoms comprises logging one or more of pain intensity, frequency, or duration.

5. The method of claim 4, wherein the interoceptive exposure comprises somatic tracking and/or provocative testing.

6. The method of claim 4, wherein the interoceptive exposure comprises one or more or more of standardized scales or visual analytics including trend graphs and heat maps to help users and clinicians identify pain triggers and understand pain progression, wherein the standardized scales comprise one of the Visual Analog Scale or the Numeric Rating Scale.

7. The method of claim 4, wherein the interoceptive exposure comprises provocative testing, and wherein the provocative testing component of the interoceptive exposure includes one or more of detailed instructions, video demonstrations, or prompts for real-time symptom feedback to facilitate reproducibility of pain triggers.

8. The method of claim 1, wherein the monitoring and recording physical symptoms comprises monitoring a measure of heart rate variability (HRV) to track physiological responses and optimize treatment interventions, and wherein the monitored HRV data is used to tailor one or more of hypnosis, meditation, or taVNS protocols to individual physiological states.

9. The method of claim 1, wherein providing to the user behavior instructions comprises one or more of:
providing pain-relieving hypnosis sessions; or
integrating meditation practices to provoke symptoms in a controlled manner.

10. The method of claim 9, wherein providing pain-relieving hypnosis sessions comprises employing techniques including one or more of guided imagery, progressive muscle relaxation, deep breathing, or cognitive restructuring, to alter pain perception and/or promote relaxation.

11. The method according to claim 9, wherein integrating meditation practices comprise one or more of Mindfulness-Based Stress Reduction (MBSR) techniques, pranayama breathing exercises, non-directive meditation, or personalized meditation plans to promote and/or enhancing one or more of managing stress, relaxation, pain relief, safety, mental clarity, enhance treatment efficacy, or a comprehensive and integrative approach to pain management.

12. The method of claim 9, wherein the pain-relieving hypnosis sessions are based on pain management techniques comprising customized session parameters, the session parameters comprising one or more of duration, background sounds, or focus areas of the body or somatic topography tailored to the user's specific pain conditions and preferences.

13. The method of claim 1, further comprising controlling the VNS device using stimulation protocols by at least:
providing a voltage/current output pattern for a control module; and
arranging the VNS device on the user for outputting a voltage/current according to a provided output pattern,
wherein the stimulation protocols are based on one or more of clinical guidelines with real-time monitoring or individualized treatment needs by adjusting one or more of intensity, frequency, or duration.

14. The method of claim 1, further comprising:
providing a dedicated computer platform; and
configuring the computer platform by providing one or more applications running on the computer platform for providing one or more of:
monitoring and recording the user's physical symptoms;
controlling the wearable electronic vagus nerve stimulating device; or providing to the user behavior instructions.

15. The method of claim 14, wherein the computer platform comprises a mobile computer device.

16. The method of claim 14, further comprising integrating all treatment components within applications on the computer to execute one or more of:
generating personalized treatment plans;
employing algorithms to analyze interoceptive exposure data and dynamically adjusting hypnosis sessions;
control the output of the deployed VNS device;
instructing on meditation practices; or
based on user-specific needs and ongoing feedback, providing one or more of real-time instructions, feedback, or progress tracking to enhance treatment adherence and effectiveness.

17. The method of claim 14, further comprising providing one or more of push notifications, reminders, progress tracking, motivational support, or daily health tips to enhance user engagement and adherence.

18. The method of claim 14, further comprising providing a secure cloud-based system comprising the computer platform, wherein the secure cloud-based system ensures one or more of data privacy, facilitating regular updates, or allowing for remote monitoring and telehealth integration by healthcare providers.
